(19) United States
(12) Patent Application Publication
(10) Pub. No.: US 2026/0088179 A1
(43) Pub. Date: Mar. 26, 2026

(54) HEALTH FUTURE PROJECTOR

(71) Applicant: Toyota Research Institute, Inc., Los Altos, CA (US)

(72) Inventors: Laura A. Libby, San Francisco, CA (US); Nikos Arechiga, San Mateo, CA (US); Nayeli S. Bravo, Palo Alto, CA (US); Candice Hogan, Foster City, CA (US)

(73) Assignees: Toyota Research Institute, Inc., Los Altos, CA (US); Toyota Jidosha Kabushiki Kaisha, Aichi-ken (JP)

(21) Appl. No.: 18/891,812

(22) Filed: Sep. 20, 2024

(51) Int. Cl. G16H 50/30; G16H 15/00; G16H 30/40; G16H 50/20

(52) U.S. Cl. CPC G16H 50/30; G16H 15/00; G16H 30/40; G16H 50/20

(57) ABSTRACT

A method may include receiving an image of a user; receiving demographic information, health information, and behavior information associated with the user; predicting, with a mathematical model, future health information for the user based on the demographic information, the health information, and the behavior information associated with the user; determining, with a reinforcement learning model, visualizations that are most effective for the user based on the demographic information, the health information, and the behavior information; and generating, with a generative artificial intelligence model, one or more predicted future images of the user based on the image of the user, the predicted future health information, and the determined visualizations.

### TECHNICAL FIELD

The present specification relates to visualizing future health projections.

### BACKGROUND

People's individual choices and behaviors may have an effect on their long-term health. Knowledge about how people's behavior affects their future health may cause them to change their behavior in order to improve their future health outcomes. However, even if people have a general sense of how their behaviors may affect their future health, this general awareness may not have as much valence as specific visualizations relating to a person's future health. Accordingly, a need exists for a health future projector.

### SUMMARY

In one embodiment, a method may include receiving an image of a user; receiving demographic information, health information, and behavior information associated with the user; predicting, with a mathematical model, future health information for the user based on the demographic information, the health information, and the behavior information associated with the user; determining, with a reinforcement learning model, visualizations that are most effective for the user based on the demographic information, the health information, and the behavior information; and generating, with a generative artificial intelligence model, one or more predicted future images of the user based on the image of the user, the predicted future health information, and the determined visualizations.

In another embodiment, a computing device may include a processor configured to receive an image of a user; receive demographic information, health information, and behavior information associated with the user; predict, with a mathematical model, future health information for the user based on the demographic information, the health information, and the behavior information associated with the user; determine, with a reinforcement learning model, visualizations that are most effective for the user based on the demographic information, the health information, and the behavior information; and generate, with a generative artificial intelligence model, one or more predicted future images of the user based on the image of the user, the predicted future health information, and the determined visualizations.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts an illustrative computing network for implementing a health future projector, according to one or more embodiments shown and described herein;

FIG. 2 depicts the server computing device of FIG. 1, according to one or more embodiments shown and described herein;

FIG. 3 depicts a flow diagram of an illustrative method of implementing a health future projector, according to one or more embodiments shown and described herein;

FIG. 4 depicts a flow diagram of an example method of training a mathematical model, according to one or more embodiments shown and described herein;

FIG. 5 depicts a flow diagram of an example method of training a reinforcement learning model, according to one or more embodiments shown and described herein;

FIG. 6A shows an example image of a user, according to one or more embodiments shown and described herein; and

FIG. 6B shows an example projected future image of a user, according to one or more embodiments shown and described herein.

### DETAILED DESCRIPTION

The embodiments disclosed herein describe a health future projector. In particular, a system is disclosed that allows a user to enter information about their current health and daily behaviors. The system may then use a mathematical model to predict likely health outcomes for the user at various points in the future. A reinforcement learning model may use collaborative filtering to predict what types of visualizations are most likely to cause the user to change their behavior to improve their future health. A generative artificial intelligence (AI) model may then generate images, based on the predicted future health outcomes and the determined visualizations, of what the user may look like at various points in the future. Research has shown that making health information personally meaningful may make people more likely to remember and act on that information. As such, presenting a personalized future projected image of the user may cause the user to modify their behavior to improve their future health.

The user may also input various behavioral modifications (e.g., changes in diet and exercise) and different predicted future images of the user may be generated based on the modified behaviors. Accordingly, the user may be encouraged to modify their behavior in specific ways to improve their future health. In particular, seeing actual images of their future self may create a more visceral reaction and have a greater effect on modifying the user's behavior than simply being told about how their current behavior may affect their future health in a less immersive manner.

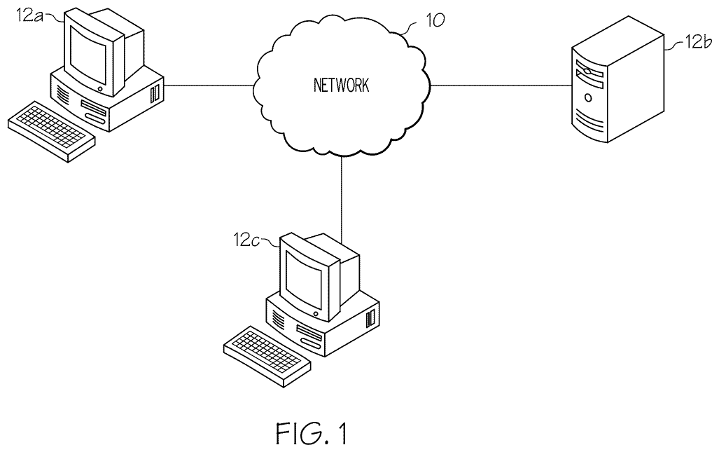

Referring now to the figures, FIG. 1 depicts an illustrative computing network, illustrating components of a system for performing the functions described herein, according to embodiments shown and described herein. As illustrated in FIG. 1, a computer network 10 may include a wide area network, such as the internet, a local area network (LAN), a mobile communications network, a public service telephone network (PSTN) and/or other network and may be configured to electronically connect a user computing device 12a, a server computing device 12b, and an administrator computing device 12c.

The user computing device 12a may be used to input information to be utilized to implement the health future projector, as disclosed herein. For example, the user computing device 12a may be a personal computer running software that a user utilizes to input demographic, health, and behavior information, as disclosed in further detail below. For example, a user may load a current image of themselves, current health information about themselves, current behavior information about themselves (e.g., information about their diet and how much they exercise), and the like. After this information is input into the user computing device 12a, the user computing device 12a or the server computing device 12b may perform the techniques disclosed herein to implement the health future projector. In some examples, the user computing device 12a may be a tablet, a smartphone, a smart watch, or any other type of computing device used by a user to input a document to be analyzed.

The administrator computing device 12c may, among other things, perform administrative functions for the server computing device 12b. In the event that the server computing device 12b requires oversight, updating, or correction, the administrator computing device 12c may be configured to provide the desired oversight, updating, and/or correction. The administrator computing device 12c, as well as any other computing device coupled to the computer network 10, may be used to input historical cost data or historical effect size data into a database.

The server computing device 12b may receive information input into the user computing device 12a by a user, and may perform the techniques disclosed herein to implement the health future projector. The server computing device 12b may then transmit generated images predicting how the user will look at one or more points in the future to be displayed by the user computing device 12a based on the operations performed by the server computing device 12b. In some examples, the server computing device 12b may be removed from the system of FIG. 1 and may be replaced by a software application on the user computing device 12a. For example, the functions of the server computing device 12b may be performed by software operating on the user computing device 12a. The components and functionality of the server computing device 12b will be set forth in detail below.

It should be understood that while the user computing device 12a and the administrator computing device 12c are depicted as personal computers and the server computing device 12b is depicted as a server, these are non-limiting examples. More specifically, in some embodiments any type of computing device (e.g., mobile computing device, personal computer, server, etc.) may be utilized for any of these components. Additionally, while each of these computing devices is illustrated in FIG. 1 as a single piece of hardware, this is also merely an example. More specifically, each of the user computing device 12a, the server computing device 12b, and the administrator computing device 12c may represent a plurality of computers, servers, databases, etc.

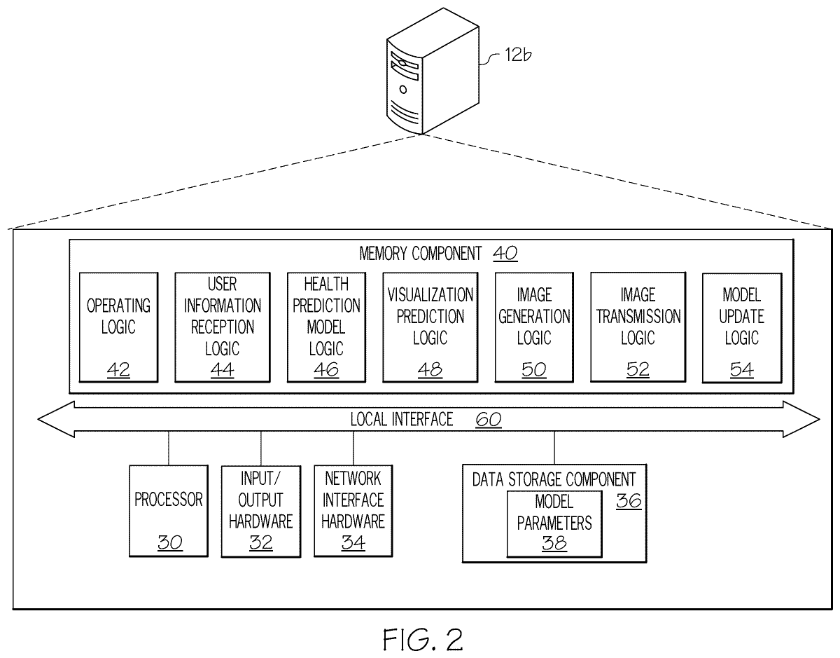

FIG. 2 depicts additional details regarding the server computing device 12b from FIG. 1. While in some embodiments, the server computing device 12b may be configured as a general purpose computer with the requisite hardware, software, and/or firmware, in other embodiments, the server computing device 12b may be configured as a special purpose computer designed specifically for performing the functionality described herein.

As also illustrated in FIG. 2, the server computing device 12b may include a processor 30, input/output hardware 32, network interface hardware 34, a data storage component 36 (which may store model parameters 38), and a non-transitory memory component 40. The memory component 40 may be configured as volatile and/or nonvolatile computer readable medium and, as such, may include random access memory (including SRAM, DRAM, and/or other types of random access memory), flash memory, registers, compact discs (CD), digital versatile discs (DVD), and/or other types of storage components. Additionally, the memory component 40 may be configured to store operating logic 42, user information reception logic 44, health prediction model logic 46, visualization prediction logic 48, image generation logic 50, image transmission logic 52, and model update logic 54 (each of which may be embodied as a computer program, firmware, or hardware, as an example). A local interface 60 is also included in FIG. 2 and may be implemented as a bus or other interface to facilitate communication among the components of the server computing device 12b.

The processor 30 may include any processing component configured to receive and execute instructions (such as from the data storage component 36 and/or memory component 40). The input/output hardware 32 may include a monitor, keyboard, mouse, printer, camera, microphone, speaker, touch-screen, and/or other device for receiving, sending, and/or presenting data. The network interface hardware 34 may include any wired or wireless networking hardware, such as a modem, LAN port, wireless fidelity (Wi-Fi) card, WiMax card, mobile communications hardware, and/or other hardware for communicating with other networks and/or devices.

It should be understood that the data storage component 36 may reside local to and/or remote from the server computing device 12b and may be configured to store one or more pieces of data for access by the server computing device 12b and/or other components. As illustrated in FIG. 2, the data storage component 36 may store the model parameters 38 of the mathematical model, reinforcement learning model, and generative artificial intelligence model, described in further detail below.

Included in the memory component 40 are the operating logic 42, the user information reception logic 44, the health prediction model logic 46, the visualization prediction logic 48, the image generation logic 50, the image transmission logic 52, and the model update logic 54. The operating logic 42 may include an operating system and/or other software for managing components of the server computing device 12b.

The user information reception logic 44 may receive user information entered by a user into the user computing device 12a. In order to predict future health outcomes for a user, the server computing device 12b may utilize current data about the user. The user information may include one or more images or videos of the user. The server computing device 12b may utilize these images or videos to generate future projected images of the user, as disclosed in further detail below.

The user information may also include demographic information associated with the user. The demographic information may include a location of the user (e.g., the user's home address, the user's work address, etc.). The server computing device 12b may utilize this location information to determine environmental factors that may affect the user's future health, as disclosed in further detail below. The demographic information may also include the age, gender, race, or other demographic information associated with the user.

The user information may also include current health information about the user. For example, the user information may include a height and weight of the user. The health information may include health related data about the user, such as body fat percentage, cholesterol, blood pressure, and the like. The health information may include medical history of the user, including any current or past health conditions suffered by the user (e.g., diseases, injuries, surgeries, and the like). The health information may include family medical history and/or genetic information associated with the user. The health information may include any medication regularly taken by the user. The health information may include information about the user's current and/or past mental health conditions (e.g., any diagnosed mental health conditions).

The user information may include behavior information about the user. In embodiments, the behavior information may include information about the user's diet. The behavior information may include exercise information about the user (e.g., the type, frequency, duration, and intensity of exercise regularly performed by the user). The behavior information may include the type of work that the user does and/or prior work history of the user. The behavior information may include information about the type of hobbies or activities the user regularly engages in.

The user information may include information about the user's social network and/or social interactions. For example, the user information may include information about the type, frequency, and emotional salience of social interactions. The user information may include whether or not the user is married. While specific examples of the user information that may be received by the user information reception logic 44 are given above, including examples of demographic information, health information, and behavior information, it should be understood that these are merely exemplary, and other types of data or information may be input by the user and used by the various models described below.

As discussed above, a user may enter user information into the user computing device 12a. In some examples, the user computing device 12a and/or the server computing device 12b may pull user information from other sources. In some examples, the user computing device 12a and/or the server computing device 12b may retrieve user information from publicly available databases (e.g., government records). In some examples, the user computing device 12a and/or the server computing device 12b may retrieve user information from private databases (e.g., from employment records, health records, a user's social media profiles, and the like). After the user computing device 12a receives the user information, either directly from the user or from other sources, the user computing device 12a may transmit the user information to the server computing device 12b. The user information reception logic 44 may cause the server computing device 12b to receive and store the user information.

Referring still to FIG. 2, the health prediction model logic 46 may predict the future health of the user using a mathematical model, as disclosed herein. As discussed above, the user information reception logic 44 may cause the server computing device 12b to receive user information associated with a user. The health prediction model logic 46 may then make future health predictions about the user based on the received user information, as disclosed herein. In the illustrated example, the mathematical model utilized by the health prediction model logic 46 to predict future health outcomes is a machine learning model. For example, a machine learning model may be trained to receive current health information for a user and make future health predictions about the user. In other examples, other types of mathematical models may be used by the health prediction model logic 46 to predict future health of the user.

Such a machine learning model may be trained using training data comprising long term health data from a large collection of individuals. The training data may comprise demographic, health, and behavior data from individuals of different ages, genders, races, and other demographic categories. The training data may comprise health data associated with individuals at different stages of life. As such, the machine learning model may be trained to predict how an individual's current health and behavior is likely to affect the individual's future health. The types of health and behavior data of individuals in the training data set may be similar to the types of health and behavior data received by the user information reception logic 44, as discussed above. The machine learning model may be trained on the data using supervised learning techniques. After the model is trained, the learned model parameters may be stored in the data storage component 36.

After the mathematical model has been trained, the health prediction model logic 46 may input the user information received by the user information reception logic 44 into the trained machine learning model. The trained model may then output predicted future health outcomes of the user based on the input user information. In particular, the model may output health information associated with the user at a variety of future time points (e.g., 5 years in the future, 10 years in the future, 20 years in the future, 30 years in the future, and the like).

The model may output a variety of different types of health information associated with the user at the various future time points. For example, the model may output a predicted life expectancy of the user, a predicted weight of the user, a predicted risk of various diseases, predicted social-emotional well-being of the user, and the like.

Referring still to FIG. 2, the visualization prediction logic 48 may predict the type of visualization that is most likely to cause the user to modify their behavior to improve their future health, as disclosed herein. As discussed above, research has shown that making health information personally meaningful may make people more likely to remember and act on that information. Accordingly, as discussed above, the server computing device 12b may generate one or more images showing what the user may look like in the future based on their current health and behavior information.

However, there are a variety of different types of visualizations that may be used to present predicted future images of the user. For example, different visual styles may be used, different angles or perspectives of the user may be used, different features of the user may be emphasized, and the like. Certain visualizations may be more likely than others to cause the user to actual change their behavior to improve their future health. Accordingly, the visualization prediction logic 48 may predict which types of visualizations of future images of the user or more likely to encourage or cause the user to modify their behavior to improve their future health, as disclosed herein.

In embodiments, the visualization prediction logic 48 may comprise a reinforcement learning model that may be trained to predict the types of visualizations that are most likely to influence a user to modify their behavior to improve their future health. The reinforcement learning model may be trained using training data collected from a large number of individuals. In one example, training data may be collected by showing a large number of individuals different types of visualizations indicating their predicted future health conditions and monitoring their behavior over time. When a person's behavior improves over time (e.g., becomes healthier) after being shown a certain type of visualization, the type of visualization shown to the person may receive a positive reward during training of the reinforcement learning model. When a person's behavior does not improve over time after being shown a certain type of visualization, the type of visualization shown to the person may be given no reward or a negative reward during training of the reinforcement learning model. As such, over time, the reinforcement learning model may be trained to learn visualizations that are more likely to cause a user to change their behavior in a positive way to improve their future health.

In some examples, instead of monitoring a person's behavior over time, the reinforcement learning model may be trained with other types of training data. For example, different people may be shown different types of visualizations of their future health and they may then be asked (e.g., via survey questions) how likely the visualization is to affect their behavior. The reinforcement learning model may then be trained based on whether each visualization received a positive or negative response from the user.

In some examples, the reinforcement learning model may use collaborative filtering. For example, the reinforcement learning model may learn different types of visualizations that are more effective at encouraging healthy behavior by different types of people. For example, the reinforcement learning model may determine N different groups of people and may learn a particular visualization that is most effective in encouraging each group to engage in healthy behavior. The visualization prediction logic 48 may then determine which of the N groups people is most likely to encourage the user to engage in healthy behaviors.

After the reinforcement learning model described above is trained, the visualization prediction logic 48 determines which group of people the user is most similar to, based on the user information received by the user information reception logic 44, and determine that the types of visualizations associated with that group of people.

The parameters of the model may be stored in the data storage component 36. The visualization prediction logic 48 may then use the trained model to predict the types of visualizations most likely to encourage the user to change their behavior to improve their future health. In some examples, the visualization prediction logic 48 may determine the types of visualization prompts to be input to a generative AI model, as disclosed in further detail below.

Referring still to FIG. 2, the image generation logic 50 may generate one or more predicted future images of a user that indicate the user's predicted future health, as disclosed herein. In the illustrated example, the image generation logic 50 utilizes a generative AI model to generate one or more predicted future images of the user. A generative AI model may receive a prompt as input and may output content based on the prompt. A text-based generative AI model (e.g., ChatGPT, Gemini) may receive a text prompt as input, and may output a text response. An image-based AI model (e.g., Midjourney, DALL-E) may receive a text prompt as input, and may output an image as a response. Generative AI models may be trained on large data sets to produce a response based on an input prompt. In the illustrated example, the generative AI model maintained by the server computing device 12b may be trained to generate images In the illustrated example, the image generation logic 50 may utilize a generative AI model to generate one or more predicted future images of the user based on the user information received by the user information reception logic 44, the health prediction made by the health prediction model logic 46, and the prompt or type of prompt generated by the visualization prediction logic 48. For example, the user information reception logic 44 may receive a current image of the user, the health prediction model logic 46 may predict a weight of the user 10 years in the future, and the visualization prediction logic 48 may generate a prompt to be input to the generative AI model to cause the generative AI model to generate a predicted image of the user 10 years in the future with the predicted weight. The image generation logic 50 may then input the generated prompt into the generative AI model, which may generate an image of what the user may look like in 10 years at the predicted weight.

While the above example generates a predicted future image of the user at a particular weight, in other examples, images of the user may be generated to indicate any number of health conditions. Furthermore, while the above example generates a predicted image of the user 10 years in the future, in other examples, images of the user may be generated at any future time. In some examples, the image generation logic 50 may generate predicted images of the user at multiple points in the future (e.g., in 5 years, 10 years, 15 years, 20 years, 25 years, 30 years, etc.).

In the above example, the image generation logic 50 generates one or more future predicted images of the user. However, in some examples, the image generation logic 50 may also generate a text description of what the user's day-to-day life is predicted to be like in the future based on the predicted future health information. For example, the image generation logic 50 may input different prompts into a text-based generative AI model to cause the model to output a text description of what the user's life may be like at different points in the future (e.g., medications that the user may have to take, limitations on physical activity of the user, chronic conditions the user may suffer, and the like).

Referring still to FIG. 2, the image transmission logic 52 may transmit the images and/or text descriptions generated by the image generation logic 50 to the user computing device 12a. The user computing device 12a may receive the generated images and/or text descriptions and display them to the user.

In some examples, the user computing device 12a may include a user interface that allows the user to browse through different images at different time periods. For example, the user interface of the user computing device 12a may receive predicted future images of the user at one-year intervals (e.g., in 1 year, 2 years, 3 years, etc.). In this example, the user computing device 12a may include a user interface that allows the user to browse through the images received by the user computing device 12a so that the user can easily view how their health may progress as different intervals into the future.

Referring still to FIG. 2, the model update logic 54 may receive updated user information, and may update the models described above with the updated user information, as disclosed herein. In particular, in some examples, the user interface of the user computing device 12a may allow the user to modify the user information after the images are displayed. For example, the user may view images of their predicted future self based on their current behavior, as described above. The user may then enter modified behavioral information to see how the predicted future information would change based on the modified behaviors.

For example, the user may initially enter information about their current diet, and the amount of exercise they typically perform, and predicted future images of the user may be generated using the techniques described above. The user may then enter different hypothetical behavior information (e.g., a healthier diet and/or more exercise). The user computing device 12a may then transmit the modified behavioral information to the server computing device 12b. The model update logic 54 may then cause the health prediction model logic 46 to be generate updated future health predictions based on the modified behavioral information. The model update logic 54 may then cause the image generation logic 50 to generate updated predicted future images of the user based on the updated future health predictions. This may allow the user to view one set of images of their future self based on their current behaviors, and another set of images of how their future self may change if they modify their behaviors. This may encourage the user to engage in healthier behaviors to improve their future health. In some examples, the user interface of the user computing device 12a may allow the user to view the generated images in augmented reality or virtual reality or compatible mobile or wearable devices.

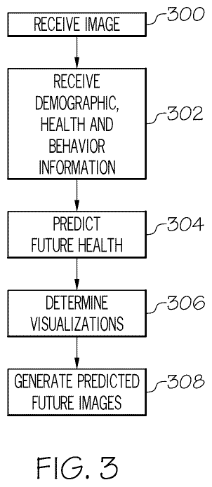

FIG. 3 shows a flowchart of an example method that may be performed by the server computing device 12b to implement a health future projector. Although the steps associated with the steps of FIG. 3 will be described as being separate tasks, in other embodiments, the blocks may be combined or omitted.

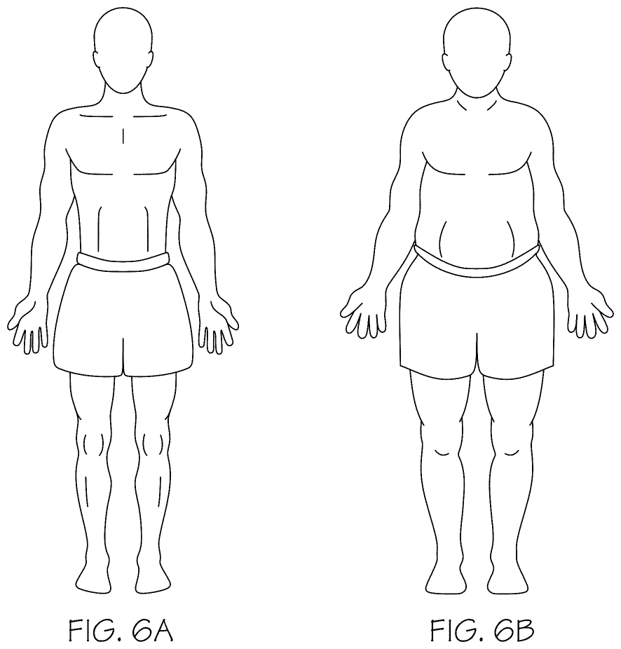

At step 300, the user information reception logic 44 receives one or more images of a user. The images may include one or more recent or current images of the user. The images may be taken from a variety of perspectives of the user. For example, FIG. 6A shows an example current image of the user. The image of user in FIG. 6A may be captured by the user (e.g., using a camera), and input through a user interface of the user computing device 12a. The user computing device 12a may then transmit the image of the user to the server computing device 12b, where it may be received by the user information reception logic 44.

Referring back to FIG. 3, at step 302, the user information reception logic 44 receives demographic information, health information, and behavior information associated with the user. As discussed above, the demographic information may include an age, gender, race, and other information about the user. The health information may include a weight of the user, medications the user is taking, any history of diseases the user has had, vital sign information such as blood pressure, blood work such as a cholesterol level, and the like. The behavior information may include information about a diet of the user, how often and what type of exercise the user engages in, and the like.

At step 304, the health prediction model logic 46 predicts future health information for the user. In particular, the health prediction model logic 46 may utilize a mathematical model to predict future health information associated with the user based on the received demographic information, health information, and behavior information. The health prediction model logic 46 may predict future health information associated with the user at a variety of times in the future. In one example, the health prediction model logic 46 may predict that the user's current diet and exercise regimen are likely to cause the user to gain weight at some point in the future.

At step 306, the visualization prediction logic 48 determines visualizations that are most effective for the user. In particular, the visualization prediction logic 48 may determine visualizations that are most likely to cause the user to change their behavior in ways that are likely to improve their future health. The visualization prediction logic 48 may utilize a reinforcement learning model to determine the most effective types of visualizations based on the received demographic information, health information, and behavior information. In some examples, the visualization prediction logic 48 generates a prompt that may be entered into a generative artificial intelligence model to generate the appropriate types of visualizations.

At step 308, the image generation logic 50 generates one or more predicted future images of the user. In particular, the image generation logic 50 may utilize a generative artificial intelligence model to generate the one or more predicted future images of the user based on the received images of the user, the predicted future health information, and the determined visualizations. In embodiments, the image generation logic 50 may input a prompt into a generative artificial intelligence model to cause the generative artificial intelligence model to generate one or more predicted future images of the user having the type of visualization determined by the visualization prediction logic 48 and indicating the health of the user predicted by the health prediction model logic 46. In some examples, the image generation logic 50 may input the prompt determined by the visualization prediction logic 48 into a generative artificial intelligence model. In one example, the image generation logic 50 may generate a future image of the user having gained weight based on a future health prediction that the user is predicted to gain weight, as shown in FIG. 6B.

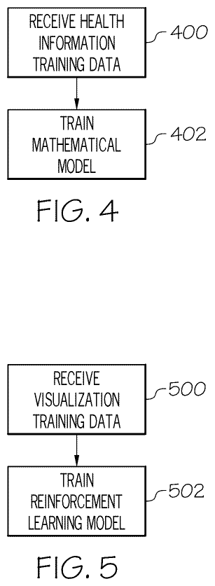

FIG. 4 shows a flowchart of an example method that may be performed to train the mathematical model maintained by the server computing device 12b, and used by the health prediction model logic 46 to predict future health of users. Although the steps associated with the steps of FIG. 4 will be described as being separate tasks, in other embodiments, the blocks may be combined or omitted.

At step 400, the server computing device 12b, or another computing device used to train the model, may receive health information training data. The health information may include demographic information, health information, and behavior information associated with a plurality of users at a plurality of different time periods. At step 402, the server computing device 12b or another computing device may use supervised learning techniques to train the mathematical model, using the health information training data, to receive current demographic information, health information, and behavior information, and predict future health information.

FIG. 5 shows a flowchart of an example method that may be performed to train the reinforcement learning model maintained by the server computing device 12b, and used by the visualization prediction logic 48 to predict visualizations that are most likely to cause users to engage in healthier behaviors. Although the steps associated with the steps of FIG. 5 will be described as being separate tasks, in other embodiments, the blocks may be combined or omitted.

At step 500, the server computing device 12b, or another computing device used to train the model, may receive visualization training data. In one examples, the visualization training data may include data indicating different types of visualizations presented to a plurality of users, and how those users changed their behaviors over time after seeing the different types of visualizations. In other examples, the visualization training data may include data indicating different types of visualizations presented to a plurality of users, and survey results indicating how likely different users are to change their behavior after seeing the different types of images. The visualization training data may also include demographic information, health information, and behavior information associated with the users.

At step 502, the server computing device 12b or another computing device may use reinforcement learning techniques to train the reinforcement learning model, using the visualization training data, to receive current demographic information, health information, and behavior information associated with a user, and predict the types of visualizations that are most likely to cause the user to change their behavior in ways likely to improve their future health. In some examples, the reinforcement learning model may be trained using collaborative filtering.

It should now be understood that embodiments described herein are directed to a health future projector. By presenting predicted future images of a user indicating how their future health may be affected by their current behavior using particular visualizations, the user may be encouraged to engage in healthier behaviors. As such, their future health may be improved.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method comprising:
receiving an image of a user;
receiving demographic information, health information, and behavior information associated with the user;
predicting, with a mathematical model, future health information for the user based on the demographic information, the health information, and the behavior information associated with the user;
determining, with a reinforcement learning model, visualizations that are most effective for the user based on the demographic information, the health information, and the behavior information; and
generating, with a generative artificial intelligence model, one or more predicted future images of the user based on the image of the user, the predicted future health information, and the determined visualizations.

2. The method of claim 1, wherein the demographic information includes an age and gender of the user.

3. The method of claim 1, wherein the health information includes a height and weight of the user.

4. The method of claim 1, wherein the health information includes medical history of the user.

5. The method of claim 1, wherein the behavior information includes diet and exercise habits associated with the user.

6. The method of claim 1, wherein the mathematical model is trained, using training data comprising demographic information, health information, and behavior information associated with a plurality of users, to receive the demographic information, the health information, and the behavior information associated with the user as input, and output the predicted future health information.

7. The method of claim 1, wherein the reinforcement learning model is trained based on training data indicating which types of visualizations have caused users to change their behavior such that their future health improved.

8. The method of claim 1, wherein the determined visualizations indicate one or more visual styles of the user.

9. The method of claim 1, wherein the determined visualizations indicate perspectives of the user.

10. The method of claim 1, wherein the determined visualizations indicate one or more features of the user.

11. The method of claim 1, wherein the reinforcement learning model is trained using collaborative filtering.

12. The method of claim 1, further comprising determining a prompt to cause the generative artificial intelligence model to generate the one or more predicted future images of the user based on the determined visualizations.

13. The method of claim 1, further comprising:
displaying the one or more predicted future images to the user;
receiving revised behavior information associated with the user;
predicting, with the mathematical model, revised future health information for the user based on the demographic information, the health information, and the revised behavior information; and
generating, with the generative artificial intelligence model, one or more revised predicted future images of the user based on the image of the user, the revised future health information, and the determined visualizations.

14. A computing device comprising a processor configured to:
receive an image of a user;
receive demographic information, health information, and behavior information associated with the user;
predict, with a mathematical model, future health information for the user based on the demographic information, the health information, and the behavior information associated with the user;
determine, with a reinforcement learning model, visualizations that are most effective for the user based on the demographic information, the health information, and the behavior information; and
generate, with a generative artificial intelligence model, one or more predicted future images of the user based on the image of the user, the predicted future health information, and the determined visualizations.

15. The computing device of claim 14, wherein the behavior information includes diet and exercise habits associated with the user.

16. The computing device of claim 14, wherein the mathematical model is trained, using training data comprising demographic information, health information, and behavior information associated with a plurality of users, to receive the demographic information, the health information, and the behavior information as input, and output the predicted future health information.

17. The computing device of claim 14, wherein the reinforcement learning model is trained based on training data indicating which types of visualizations have caused users to change their behavior such that their future health improved.

18. The computing device of claim 14, wherein the reinforcement learning model is trained using collaborative filtering.

19. The computing device of claim 14, wherein the processor is further configured to determine a prompt to cause the generative artificial intelligence model to generate the one or more predicted future images of the user based on the determined visualizations.

20. The computing device of claim 14, wherein the processor is further configured to:
display the one or more predicted future images to the user;
receive revised behavior information associated with the user;
predict, with the mathematical model, revised future health information for the user based on the demographic information, the health information, and the revised behavior information; and
generate, with the generative artificial intelligence model, one or more revised predicted future images of the user based on the image of the user, the revised future health information, and the determined visualizations.
